(19) United States
(12) Patent Application Publication
(10) Pub. No.: US 2017/0209225 A1
(43) Pub. Date: Jul. 27, 2017

(54) STEREOTACTIC MEDICAL PROCEDURE USING SEQUENTIAL REFERENCES AND SYSTEM THEREOF

(71) Applicant: Danling Wu, Cypress, CA (US)

(72) Inventor: Danling Wu, Cypress, CA (US)

(21) Appl. No.: 15/482,951

(22) Filed: Apr. 10, 2017

(51) Int. Cl. A61B 34/20; A61B 6/12; A61B 6/03

(52) U.S. Cl. CPC A61B 34/20; A61B 6/032; A61B 6/12

(57) ABSTRACT

The present invention provides a stereotactic medical procedure performed on an anatomy of a patient, and a system used for the procedure. Two or more references such as fiducial markers are used sequentially or consecutively in the medical procedure. In an embodiment, the invasive affixation of those references and the invasive portion of the medical procedure can be consolidated in a short and compact operation. The trauma and pain suffered by a patient is thus alleviated. Moreover, the present invention can expand the application of image guiding technique to many medical procedures that would otherwise preclude such application.

### CROSS-REFERENCE TO RELATED APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### FIELD OF THE INVENTION

The present invention generally relates to a stereotactic medical procedure performed on an anatomy of a patient, and a system used for the procedure. Although the invention will be illustrated, explained and exemplified by a surgical navigation system and an image guided procedure that tracks both a portion of a patient's anatomy such as jaw bone and an instrument such as a dental drill, relative to a navigation base such as image data, it should be appreciated that the present invention can also be applied to other fields, for example, physiological monitoring, guiding the delivery of a medical therapy, and guiding the delivery of a medical device, an orthopedic implant, or a soft tissue implant in an internal body space.

### BACKGROUND OF THE INVENTION

Stereotactic surgery is a minimally invasive form of surgical intervention, in which a three-dimensional coordinate system is used to locate targets inside the patient's body and to perform on them some action such as drilling, ablation, biopsy, lesion, injection, stimulation, implantation, and radiosurgery (SRS). Plain X-ray images (radiographic mammography), computed tomography, and magnetic resonance imaging can be used to guide the procedure. Stereotactic surgery works on the basis of three main components: (1) a computer based stereotactic planning system, including atlas, multimodality image matching tools, coordinates calculator, etc.; (2) a stereotactic device or apparatus; and (3) a stereotactic localization and placement procedure.

For example, in an image-guided surgery, the surgeon utilizes tracked surgical instruments in conjunction with preoperative or intraoperative images in order to indirectly guide the procedure. Image guided surgery systems use cameras or electromagnetic fields to capture and relay the patient's anatomy and the surgeon's precise movements in relation to the patient, to computer monitor in the operating room.

Real time image guided surgery has been introduced into dental and orthopedic area for years. Typically, a system includes a treatment planning software, a marker system attached to patient's anatomy, a 3D camera system to track the markers, a registration software module to align the actual patient position with the patient image in the treatment plan, a software module to display the actual surgical tool positions and the planned positions on the computer screen.

The most important part of the system is the fiducial markers and the marker tracking system. In principle, the fiducial markers must be placed onto the patient's anatomy before surgery and during the surgery. The relative positions between the markers and the surgery site must be fixed. For example, in a dental implant placement system, if the doctor is going to place implants on the lower jaw, the markers have to be placed on the lower jaw, and they shall not move in the process. If the markers are placed onto for example the upper jaw, they are useless, because the jaws can move relative to each other all the time.

If, for any reason the markers cannot be placed in the same locations, the image guided surgery could not be performed. For example, if markers are placed onto the patient's teeth or soft tissue, when the doctor needs to extract all the teeth, or open the soft tissue, the markers must be removed, and the procedure cannot continue.

Therefore, there exists a need to overcome the aforementioned problems. Advantageously, the present invention provides a method that can perform the tracking even when the markers system cannot be placed in the same positions before and during the tracking process. The present invention provides a surgery navigation system that can support multistage markers and tracking, when the initial set of markers have to be removed or cannot be placed at an ideal position.

### SUMMARY OF THE INVENTION

One aspect of the present invention provides a stereotactic medical procedure using sequential or consecutive references. The procedure comprises the steps of (i) affixing a first reference to an anatomy of a patient, (ii) obtaining a navigation base of the patient with the first reference being affixed to the anatomy; (iii) affixing a second reference to the anatomy; (iv) registering the second reference to the first reference or to the navigation base; and (v) performing at least a part of the medical procedure guided by the navigation base using the second reference. The navigation base may be image data, a coordinate system, an atlas, a morphed atlas, or any combination thereof. Typically, the navigation base may include the first reference data, i.e. a counterpart of the first reference in the navigation base. In some embodiments, step (i) of affixing a first reference to an anatomy of a patient is carried out before an imaged guided surgery. After step (ii) of obtaining the navigation base, the first reference is removed from the anatomy. At the beginning of the surgery, the first reference is affixed back to the anatomy as it was in step (i), and registered to the first reference data in the navigation base (i.e. the counterpart of the first reference in the navigation base). During the surgery, step (iv) is implemented.

In the present invention, two or more references such as fiducial markers are used sequentially or consecutively in the medical procedure. In an embodiment, the invasive affixation of those references and the invasive portion of the medical procedure can be consolidated in a short and compact operation. The trauma and pain suffered by a patient is thus alleviated.

Another aspect of the invention provides a system used for the aforementioned stereotactic medical procedure. The system includes (1) two or more references for affixing to an anatomy of a patient sequentially or consecutively; (2) a navigation base acquisition system for obtaining a navigation base of the patient with a first reference selected from the two or more references being affixed to the anatomy; (3) a planning module for planning the stereotactic medical procedure; (4) a foreign object such as a surgical tool, which is defined as an object that is foreign to the anatomy, but will move relative to the anatomy for the purpose of executing the stereotactic medical procedure; (5) a tracking system for tracking both the anatomy and the foreign object; (6) a translation module for registering the data obtained by the tracking system into the planning module; (7) a superimposing module for overlaying the anatomy and the foreign object as tracked by the tracking system; and (8) a cascading module for shifting the reliance on one reference to the reliance on another, during the stereotactic medical procedure. The system may also include (9) a displaying module for calculating and displaying the relative positions of the foreign object and the anatomy.

The above features and advantages and other features and advantages of the present invention are readily apparent from the following detailed description of the best modes for carrying out the invention when taken in connection with the accompanying drawings.

### BRIEF DESCRIPTION OF THE SEVERAL VIEWS OF THE DRAWINGS

The present invention is illustrated by way of example, and not by way of limitation, in the figures of the accompanying drawings and in which like reference numerals refer to similar elements. All the figures are schematic and generally only show parts which are necessary in order to elucidate the invention. For simplicity and clarity of illustration, elements shown in the figures and discussed below have not necessarily been drawn to scale. Well-known structures and devices are shown in simplified form, omitted, or merely suggested, in order to avoid unnecessarily obscuring the present invention.

FIG. 1 schematically shows a system used for the stereotactic medical procedure in accordance with an exemplary embodiment of the present invention.

FIG. 2 illustrates a stereotactic medical procedure using sequential or consecutive references in accordance with an exemplary embodiment of the present invention.

FIG. 3 illustrates another stereotactic medical procedure using sequential or consecutive references in accordance with an exemplary embodiment of the present invention.

FIG. 4 demonstrates still another stereotactic medical procedure using sequential or consecutive references in accordance with an exemplary embodiment of the present invention.

FIG. 5 depicts yet another stereotactic medical procedure using sequential or consecutive references in accordance with an exemplary embodiment of the present invention.

FIG. 6 schematically shows a dental procedure using a dental drill in accordance with an exemplary embodiment of the present invention.

FIG. 7 illustrates the affixation of a first reference to the anatomy such as a gum tissue in accordance with an exemplary embodiment of the present invention.

FIG. 8 illustrates the affixation of a second reference to the anatomy such as a jawbone in accordance with an exemplary embodiment of the present invention.

FIG. 9 illustrates a flap surgery followed by an image guided dental drilling and implantation in accordance with an exemplary embodiment of the present invention.

FIG. 10 illustrates the affixation of a first reference to the anatomy such as a tooth in accordance with an exemplary embodiment of the present invention.

FIG. 11 illustrates the affixation of a second reference to the anatomy such as a jawbone in accordance with an exemplary embodiment of the present invention.

FIG. 12 illustrates the extraction of the tooth that was affixed with a first reference in accordance with an exemplary embodiment of the present invention.

FIG. 13 illustrates an image guided dental drilling and implantation into the extraction socket in accordance with an exemplary embodiment of the present invention.

### DETAILED DESCRIPTION OF THE PREFERRED EMBODIMENT

In the following description, for the purposes of explanation, numerous specific details are set forth in order to provide a thorough understanding of the present invention. It is apparent, however, to one skilled in the art that the present invention may be practiced without these specific details or with an equivalent arrangement.

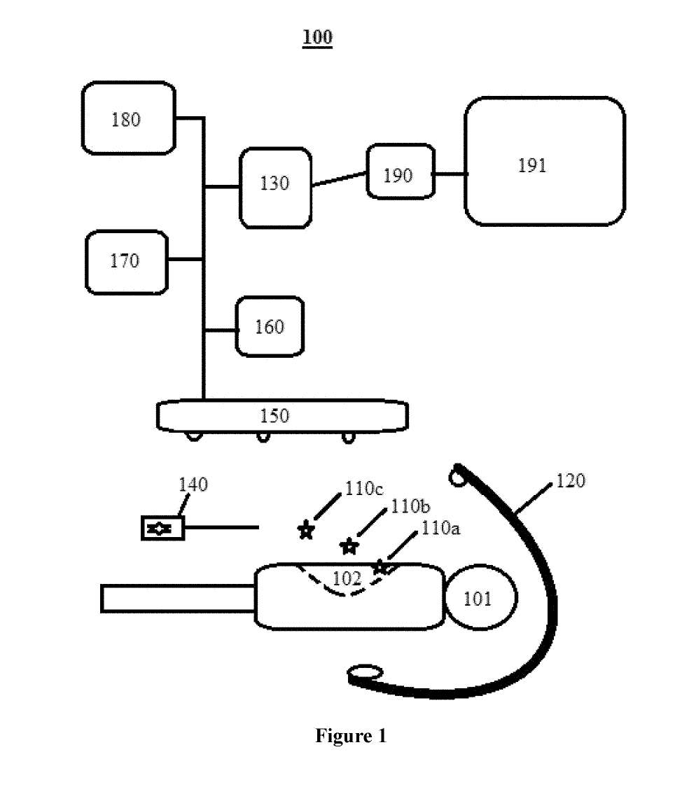

In the embodiment as shown FIG. 1, the system 100 for the stereotactic medical procedure includes two or more references 110a, 110b, 110c, and so on, which are to be affixed to an anatomy 102 of a patient 101. Examples of the “two or more references” include, but are not limited to, two or more sets of fiducial markers, conveniently named as a first set of fiducial markers, a second set of fiducial markers, a third set of fiducial markers, and so on. A set of fiducial markers may be the same as, or different from, another set of fiducial markers. One fiducial marker within any given set of fiducial markers may be the same as, or different from, another fiducial marker within that set. There is no specific limitation on the design, material, and the placement approach of the markers, as long as the shape and material of the fiducial markers can identify the position of a “marked” subject.

Referring again to FIG. 1, system 100 includes a navigation base acquisition system 120 for obtaining a navigation base of the patient with a first reference (e.g. 110a) selected from the two or more references (110a, 110b, 110c . . . ) being affixed to the anatomy 102. The navigation base may be image data, a coordinate system, an atlas, a morphed atlas, or any combination thereof. A planning module 130 is employed for planning the stereotactic medical procedure System 100 further includes a foreign object 140 that is foreign to, and moves relative to, the anatomy 102, a tracking system 150 for tracking both the anatomy 102 and the foreign object 140; a translation module 160 for registering the data obtained by the tracking system 150 into the planning module 130; a superimposing module 170 for overlaying the anatomy 102 and the foreign object 140 as tracked by the tracking system 150 with their counterparts in the planning module, and a cascading module 180 for shifting the reliance on one reference to the reliance on another, during the stereotactic medical procedure. The system may also include a displaying module 190 for calculating and displaying the relative positions of the foreign object 140 and the anatomy 102 on a display device 191.

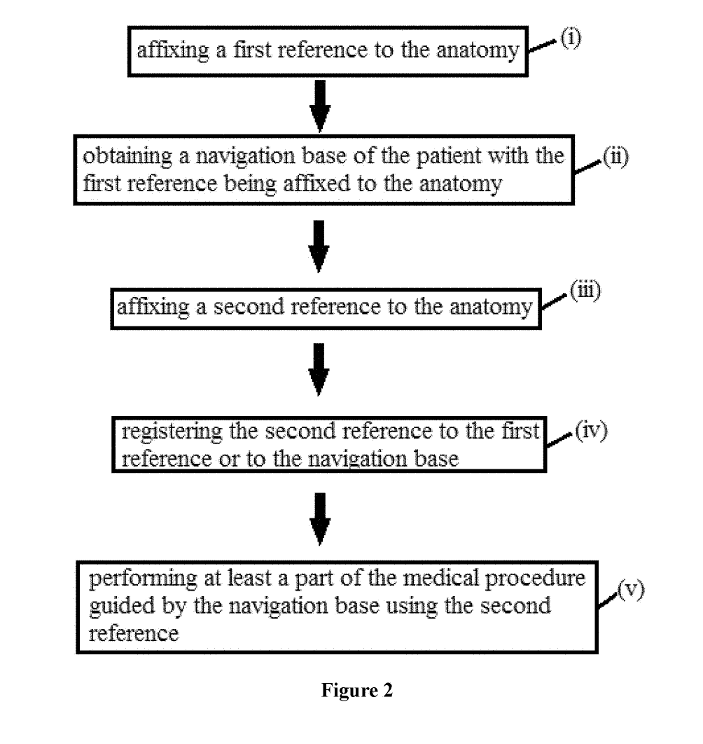

In an exemplary embodiment as shown in FIG. 2, the stereotactic medical procedure is performed on an anatomy of a patient. The procedure includes the steps of (i) affixing a first reference to the anatomy; (ii) obtaining a navigation base of the patient with the first reference being affixed to the anatomy, wherein the navigation base is image data, a coordinate system, an atlas, a morphed atlas, or any combination thereof, (iii) affixing a second reference to the anatomy; (iv) registering the second reference to the first reference or to the navigation base; and (v) performing at least a part of the medical procedure guided by the navigation base using the second reference.

Examples of the stereotactic medical procedure according to the invention include, but are not limited to, a navigated surgery; a physiological monitoring; a delivery of a medical therapy; a delivery of a medical device, an orthopedic implant, or a soft tissue implant; or any combination thereof. The patient may be a human or an animal. Examples of the anatomy include, but are not limited to, a jaw, a knee, a spine, a brain, a heart, and so on. In a specific embodiment, the anatomy is a jaw, and the stereotactic medical procedure includes a flap surgery followed by an image guided dental drilling and implantation.

It is broadly contemplated that, a first reference, a second reference, or more (if necessary) reference(s) are employed to track the anatomy of the patient where the procedure is carried out (hereinafter “anatomy of interest”). Since movement of the anatomy of interest may occur during the procedure, the first/second reference is generally affixed to the patient in a permanent or immovable fashion. However, sometimes the first/second reference cannot be affixed to the patient in a permanent or immovable fashion, and must be temporarily removed and then re-affixed back. In this situation, the re-affixation or re-placement of the first/second reference must be precise and repeatable, as it was affixed or placed previously. If the affixation or placement of the first/second reference is precise and repeatable, it may also serve as a fiducial marker that may be used in preoperative imaging and during registration intra-operatively. As such, an example of the first reference may be a first set of fiducial markers Similarly, an example of the second reference may be a second set of fiducial markers, and so on.

A fiducial marker is an object placed in the field of view of an imaging system which appears in the navigation base produced, for use as a point of reference. For example, the fiducial markers may include materials that are substantially radio-opaque or opaque to the imaging process. Various materials may be used to form the radio-opaque fiducial markers, such as metals, chemical compounds, and various mixtures. Fiducial markers of known pattern and size can serve as real world anchors of location, orientation and scale. In preferred embodiments of the invention, these fiducial markers are visible both on pre-operative images, such as computerized tomography (“CT”) scans or magnetic resonance imaging (“MRI”), and in real-time by the surgeon by visualization or use of tracking system 150.

In steps (i) and (ii), the first set of fiducial markers may be attached or affixed to the patient during the acquisition of the navigation base, such as pre-operative images, and may remain attached or affixed to, or re-attach or re-affix to, the patient, in steps (iii) and/or step (iv). This insures that the navigation base to be accurately aligned and correlated with the anatomy of interest during the navigated procedure of the invention.

A preferred example of the first reference 110a is a first set of fiducial markers with a geometric shape or a material that can be recognized by tracking system 150, e.g. a computer vision system. The first reference is affixed non-invasively to the anatomy in step (i). Specifically, the first set of fiducial markers needs not penetrate the dermis (no incision is needed) to be fixed directly to a rigid portion of the anatomy, such as a bony portion in the patient. For example, the first set of fiducial markers may be placed on teeth, if the teeth are stable and not shaking. Alternatively, the first set of fiducial markers may be interconnected with a selected portion of soft tissue, such as a gum tissue, a skin, a muscle, a tendon, a ligament, or any other appropriate soft tissue portion. Therefore, the first set of fiducial markers can be easily fixed and removed from the patient with no trauma and pain. This enables a simple and quick positioning of the first set of fiducial markers during an operative procedure. The first set of fiducial markers may be fixed to the patient in any non-invasive manner, for example, using painted-on or sprayed-on adhesives, a fastening device, or a “double-sided” tape. In other embodiments, the first reference may be fitted onto or in a tooth cap that may be fit over a tooth, an oral bite block that may be held within the teeth or jaws of the patient or any other appropriate location.

The navigation base of the invention may be image data selected from two, three, and four dimensional images acquired from computed tomography (CT), magnetic resonance imaging (MRI), single photon emission computer tomography (SPECT), positron emission tomography (PET), ultrasound imaging (US), high frequency ultrasound (HIFU), intra-vascular ultrasound (IVUS), fluoroscopic imaging, isocentric fluoroscopy, bi-plane fluoroscopy, multi-slice computed tomography (MSCT), optical coherence tomography (OCT), or any combination thereof. Therefore, examples of the “navigation base acquisition system” include, but are not limited to, an image acquisition system, such as a CT scanner for scanning the patient with the fiducial markers.

In step (ii) of obtaining a navigation base of the patient, initial pre-operative images may be taken with the first reference 110a in place, i.e. being attached to the anatomy and used as a fiducial marker. In an embodiment, navigation base acquisition system 120 may be an imaging device that generates x-rays from an x-ray source that propagate through the patient into an x-ray receiving device. The receiving device generates an image representing the intensities of the received x-rays. Typically, the receiving device includes an image intensifier that first converts the x-rays to visible light, and a charge coupled device (CCD) video camera that converts the visible light into digital images. The receiving device may also be a digital device that converts x-rays directly to digital images, thus potentially avoiding distortion introduced by first converting to visible light.

In some embodiments, images of the same anatomy may be produced with two different imaging systems, and then be correlated by placing a fiducial marker in the area imaged by both systems. The fiducial marker must be visible in the images produced by both imaging modalities. By this method, functional information from SPECT or positron emission tomography can be related to anatomical information provided by magnetic resonance imaging (MRI). Similarly, fiducial points established during MRI can be correlated with brain images generated by magnetoencephalography to localize the source of brain activity.

In an embodiment, the planning module 130 is used for the surgical treatment planning. For example, the planning module 130 takes the data from an image acquisition system, recognizes and extracts the fiducial markers' information, and calculates the spatial relationship between the markers and the patient anatomy. The planning module 130 may plan a surgical treatment using e.g. CT data and MRI data.

The first set of fiducial markers 110a is used to assure that the navigation base of a patient, such as pre-acquired or atlas images, may be registered to the real-time anatomy of interest.

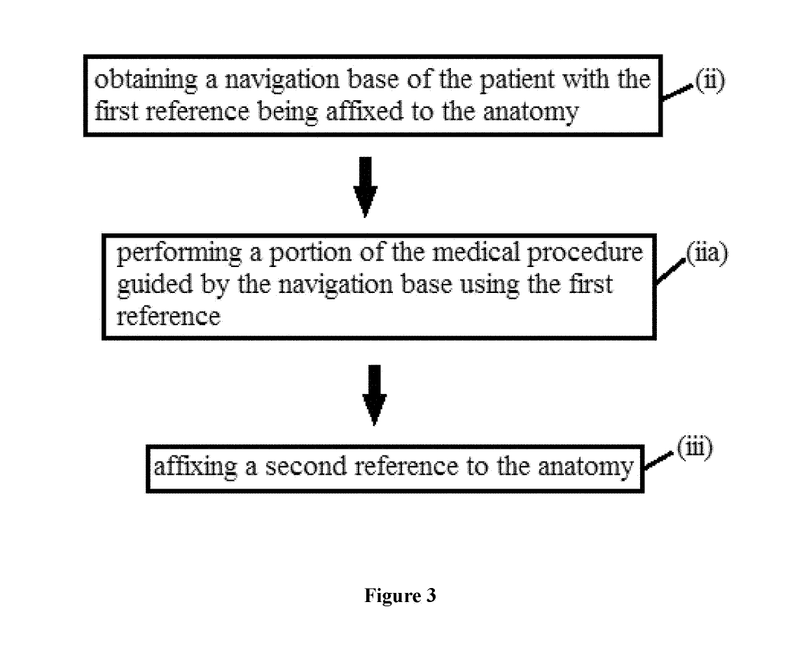

As shown in FIG. 3, the stereotactic medical procedure may optionally include a step (iia) of performing a portion of the medical procedure guided by the navigation base using the first reference that remains affixed, or is re-affixed, to the anatomy as it was in the step of obtaining the navigation base, before the step of affixing a second reference to the anatomy.

Similar to the first reference, the second reference 110b) comprises a second set of fiducial markers with a geometric shape or a material that can be recognized by tracking system 150 e.g. a computer vision system. The reason for introducing the second set of markers during the surgery may be (1) the first set of markers has to be removed during the surgery; (2) the second set of markers cannot be placed before the surgery because they have to be for example invasively placed; and/or (3) other reasons. When the second reference is affixed invasively to the anatomy in, it needs penetrate the dermis (incision is needed) to be fixed directly to a rigid portion of the anatomy, such as a bony portion in the patient.

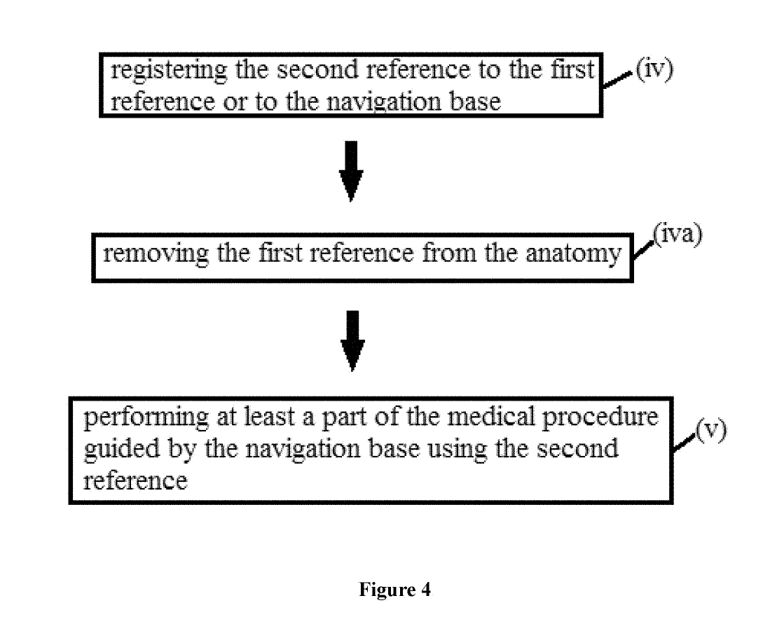

As shown in FIG. 4, the stereotactic medical procedure may further include a step of (iva) of removing the first reference 110a from the anatomy 102 after the second reference is registered to the first reference or the navigation base, and before the medical procedure guided by the navigation base is performed in step (v). In a specific embodiment, the first reference 110a is affixed to a site of the anatomy 102 in step (i), and the site is subject to the operation of step (v) in the medical procedure performed. For example, such site of the anatomy may be a gum tissue that will be flapped for later dental implantation.

Cascading module 180 is used for shifting the reliance on the first reference 110a to the reliance on the second reference 110b), during the stereotactic medical procedure. Cascading module 180 may be, for example, a component associated with the planning module 130, or a separate software module. In an embodiment, cascading module 180 can recognize both the first set fiducial markers and the second set of fiducial markers, and then find out the spatial relationship between the two sets of markers. As a result, the cascading module 180 can identify the position and orientation of the foreign object 140 such as surgical tool corresponding to the second set of fiducial markers, the position and orientation of the subject anatomy corresponding to the second set of the markers, the relations between the surgical tool and the patient anatomy, as well as the relations between the surgical tool and the treatment plan. In a similar manner, the cascading module can also process the transition from the second set of fiducial markers to the third, from the third to the fourth, and so on and so forth, if needed.

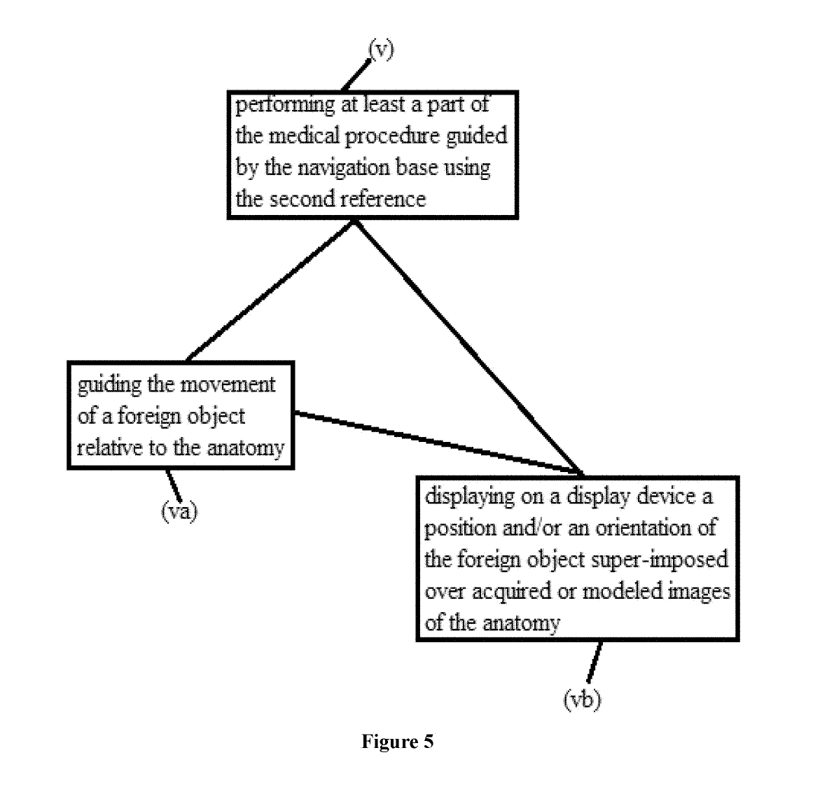

As shown in FIG. 5, during the medical procedure performed in step (v), the movement of a foreign object 140 relative to the anatomy is guided using the second reference 110b and the navigation base. Examples of the foreign object 140 include, but are not limited to, an instrument, a tool, an implant, a medical device, a delivery system, or any combination thereof. The foreign object 140 may be a dental drill, a probe, a guide wire, an endoscope, a needle, a sensor, a stylet, a suction tube, a catheter, a balloon catheter, a lead, a stent, an insert, a capsule, a drug delivery system, a cell delivery system, a gene delivery system, an opening, an ablation tool, a biopsy window, a biopsy system, an arthroscopic system, or any combination thereof.

Tracking system 150 is important for the stereotactic medical procedure of the invention. Examples of the tracking system 150 include, but are not limited to, a camera system. For example, the camera system can acquire the image and/or positions of the second reference 110b (e.g. fiducial markers) on the subject and trackable markers on the foreign object 140 e.g. a surgical tool, and send the acquired data to a computer system. In step (v) of performing the medical procedure, the navigation system must be able to detect both the position of the patient's anatomy and the position of the surgical tool. Knowing the location of these two items allows the navigation system to compute and display the position of the tool in relation to the anatomy, even both are moving. Therefore, tracking system 150 is employed to track the foreign object 140 and the anatomy 102 simultaneously. The translation module 160 may be, for example, a component associated with the planning module 130, or a separate software module. The translation module 160 can register the data obtained by the tracking system 150 such as camera system with the data in the planning module 130, and find out the correspondences between the actual subject (patient position) and the treatment.

As shown in FIG. 5, during the medical procedure performed in step (v), the position and/or orientation of the foreign object 140 superimposed over acquired or modeled images of the anatomy 102 are displayed on a display device, making “image guided” procedure possible The superimposing module 170 in FIG. 1 may be, for example, a component associated with the planning module 130, or a separate software module The superimposing module 170 can recognize the trackable markers on the surgical tool, and register them with the predefined marker data to identify the actual position of the surgical tool with respect to the second set of fiducial markers 110b and consequently to the patient anatomy 102 with substantial spatial identity and substantial spatial fidelity. The displaying module 190 may be connected to a display device 191. The displaying module 190 may be, for example, a component associated with the planning module 130, or a separate software module. The displaying module 190 calculates and displays the relative positions of the actual surgical tool 140 and the subject anatomy 102. It may show the position, trajectory and movement of the surgical tool 140 in a substantially real-time fashion on the overlay of the computer-generated reconstruction. The displaying module 190 may or may not need to display the actual shape or size of the tool and the subject anatomy. Any representations, such as texts, numbers, symbols, diagrams, or images, will work as long as their relative positioning information can be inferred from the representation.

The foreign object 140 may be tracked relative to the anatomy 102 to provide up to six degree of freedom information regarding the location of the foreign object 140 represented as 3-dimensional position (x, y, z) and 3 degrees of orientation (yaw, pitch, and roll) for guiding the movement of the foreign object 140.

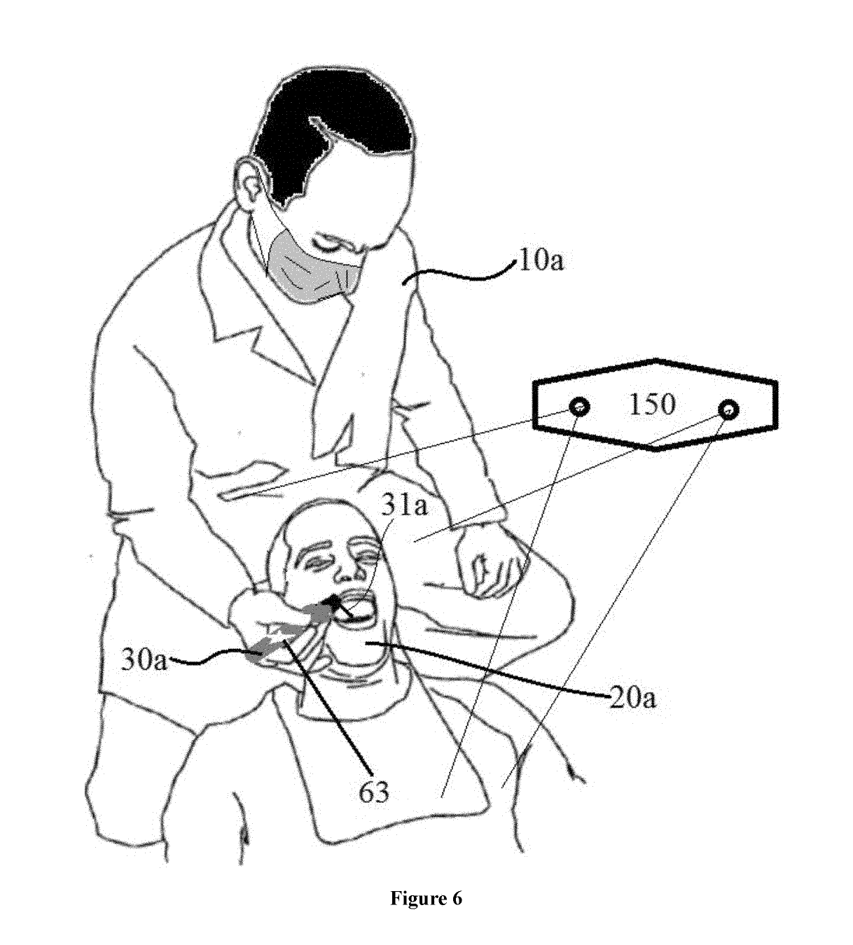

Referring to FIG. 6, dental surgeon 10a is preparing a drilled core for the placement of a dental implant on an anatomy such as a jawbone 20a with a foreign object 140 such as a dental drill system with handpiece 30a and dental drill 31a. Tracking system 150 measures the spatial relationship between reference(s) 63 on the drill system 30a and 31a and the second reference (not shown) on the jawbone 20a, to infer the spatial relationship between 30a and 31a, and jawbone 20a. The position and orientation of the handpiece 30a and the drill 31a is supplied to the tracking system 150 by means of trackable marker(s) 63 on the handpiece 30a. Trackable markers in the foreign object 140 may generally be optical detection points e.g. LEDs or reflectors, an acoustic sensor, and electromagnetic coils that may be detected by a suitable navigation system. For example, the drill system may be equipped with a number of LED emitters, whose radiation is tracked by tracking system 150. The position of these LED's may be tracked by means of a triangulation tracking and measurement technique, or any other suitable tracking and measurement technique, such that the spatial position and orientation of the handpiece 30a, particularly the drill 31a, is known at all times. Tracking system 150 should be understood broadly as including any form of sensor device operative for providing 3-D information about the position of the tracked body such as the handpiece 30a, drill 31a and jawbone 20a. Similarly, the position and orientation of the jawbone 20a being drilled is also supplied to tracking system 150 by means of the second reference 110b whose position is defined relative to the patient's jaw or jawbone.

In a representative embodiment as described in the following, a flap surgery in dental implant placement will be implemented in two stages using two sets of fiducial markers. The second set of fiducial markers can only be invasively placed during the surgery. It is broadly contemplated that an invasive placement of the second reference may involve bone screw, and an incision made through a selected portion of soft tissue, such as gum, dermis, skin, fascia, and muscle.

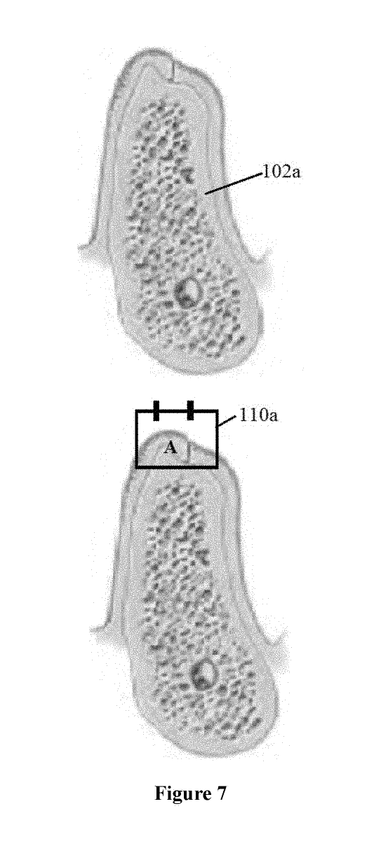

As shown in FIG. 7, in steps (i) and (ii), a first set of fiducial markers 110a is placed onto a patient's jawbone 102a for scanning and treatment planning. Before the treatment, a first set of markers 110a is placed onto patients existing anatomy at location A, as long as the position is fixed with references to the surgical site. In the flap surgery, a first set of fiducial markers is placed onto the gum when the patient is scanned and the treatment is planned according to the markers. FIG. 7 shows a fully edentulous case, in which markers 110a are placed on the arch for data acquisition and planning before the surgery. In step (ii), the patient is scanned wearing the markers 110a, then the markers are taken out. The scan data is processed in the treatment planning module so that all the operations are defined with respect to the markers 110a.

When it is the time for treatment, the first set of fiducial markers 110a is placed back where it was when the navigation base data was originally obtained, for the purpose of tracking the positions of jawbone 102a. During the surgery, the tracking system 150 is following the markers, and communicates with the planning module 130, so that the system knows where to operate. The surgery can be carried out until the doctor needs to remove markers 110a, and work on the anatomical site A where the markers 110a were placed. For example, when the gum soft tissue at location A needs to be flapped, markers 110a would obstruct the operation, and must be removed in the first place.

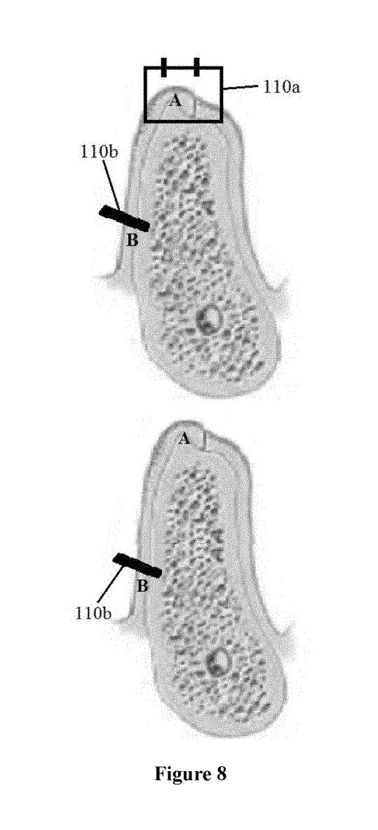

As shown in FIG. 8, in step (iii), the second set of fiducial markers 110b is placed onto the jawbone 102a during tracking time. Placing the second set of markers 110b can be guided by tracking the first set of markers 110a. In step (iv), the second set of fiducial markers 110b can be registered with the first set of fiducial markers 110a that was previously placed. The tracking of jawbone 102a continues with the second set of fiducial markers 110b, after the first set of fiducial markers 110a is removed.

Before the first set of fiducial markers 110a (as e.g. stage I markers) is removed, the second set of fiducial markers 110b (as e.g. stage II markers) can be placed onto jawbone 102a at a different location, say location B. Sometimes it is not possible to place stage II markers before the surgery. A reason may be that there is not enough space in the surgical site to place stage II markers. Another reason may be that stage II markers can only be placed invasively. For example, in a flap surgery in dental implant placement, the patient's gum needs to be opened first, and then the jaw bone is exposed and treated. Preferably, the markers are placed onto the bone structure so they remain in the same spatial relationship. Although one can place some mini implants as markers onto the bone at the planning stage, and then replace them again at the surgery time, the patients will suffer more trauma and pain, and the procedure is costly too.

In the flap surgery, the doctor may first place the first set of fiducial markers 110a onto the same location A of the gum. The doctor may then drill some holes on the patient's jaw bone 102a using the first set of markers 110a as the trackers, and insert anchor pins into the holes. The anchor pins will not appear in the image data, and users can then specify the anchor pins as stage II markers, or the second set of fiducial markers 110b. From this point of time on, the first set of markers 110a is no longer needed, and the operation can be carried out on opened gum with the stage II markers 110b, even though the first set of markers 110a are removed.

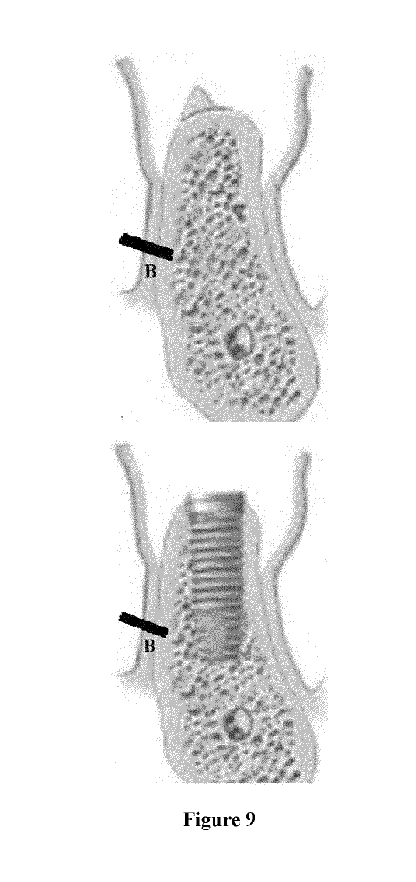

In step (iv), the camera system 150 will acquire the image of the second set of fiducial markers 110b together with that of the first set 110a. The positional relationship between the second set of fiducial markers 110b and jaw bone 102a is then resolved by combining the transformation between the first set of fiducial markers 110a and jaw bone 102a, and that between the second set of markers 110b and jaw bone 102a. From this point on, the system no longer needs the first set of fiducial markers 110a to continue the remaining treatment, because the relationship between the stage II markers 110b and the patient is defined. As shown in FIG. 9, the gum tissue at location A is opened, the guided drilling with the dental drill 30a is completed, and then the implant 88 is placed into the jaw bone 102a. Then, stage II markers 110b can be removed. The same process can continue whenever the current markers need to be removed and a new site will be used to place next markers.

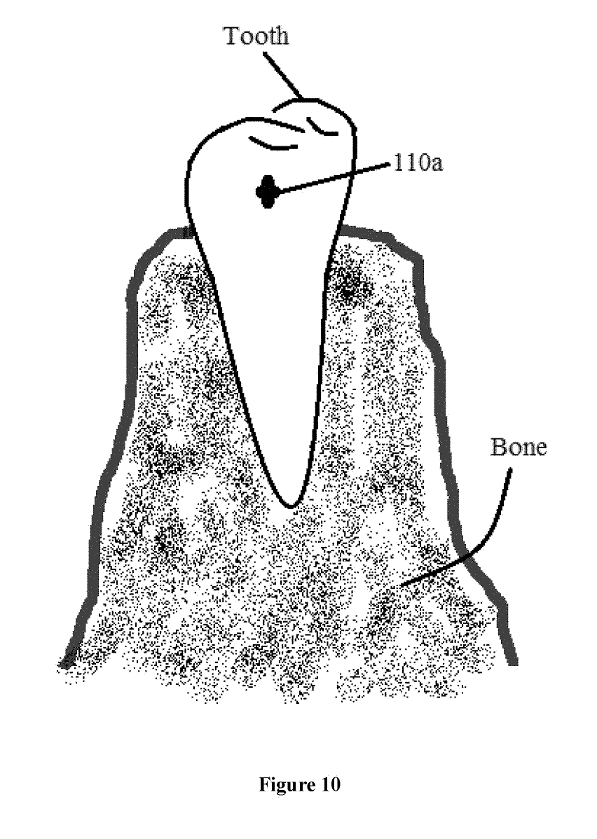

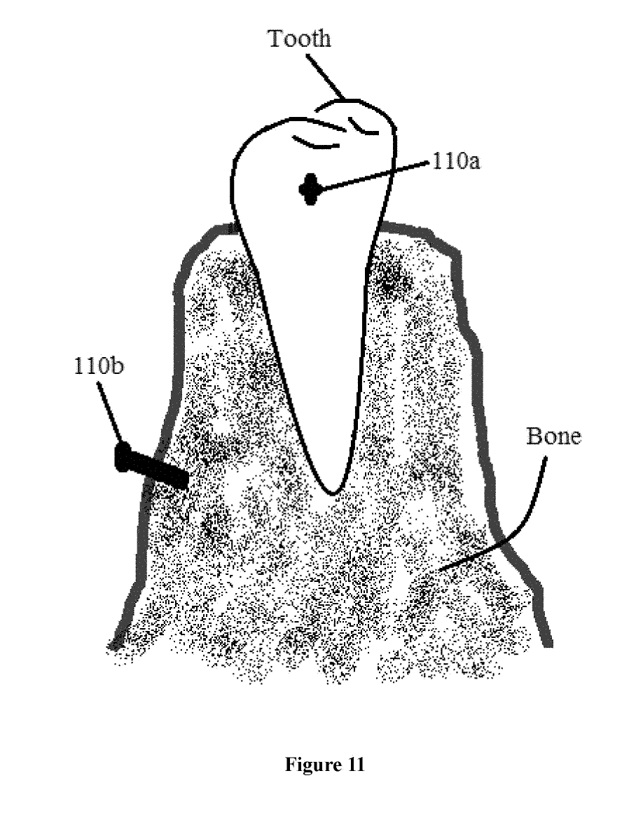

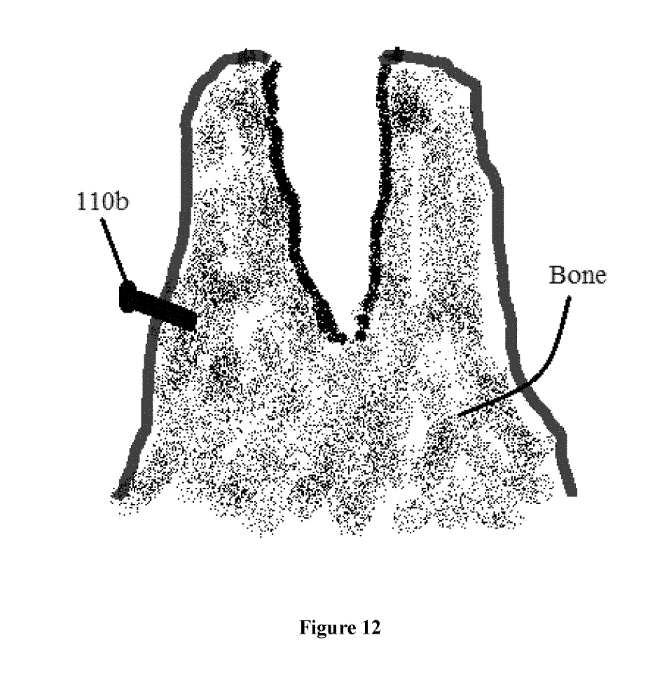

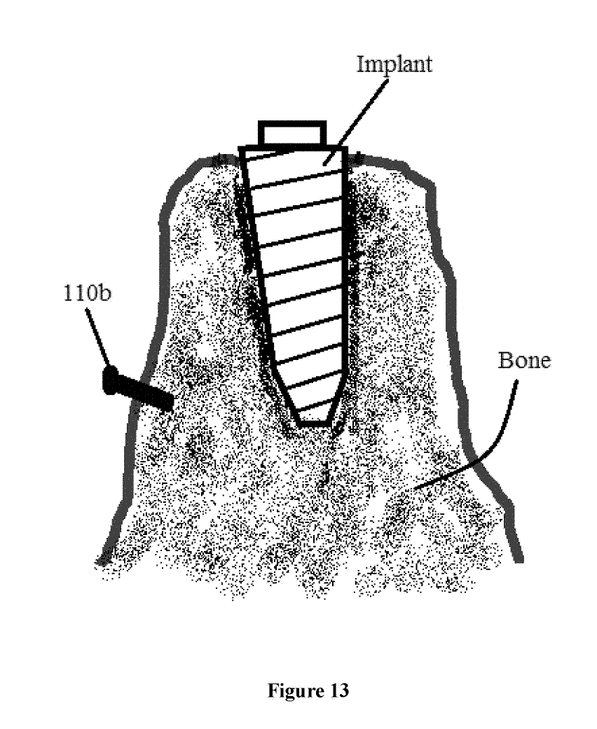

As known to a dentist, after a compromised tooth is removed, the resultant extraction socket is left to heal for four to six months before a dental implant is placed. However, marked alterations occur in the edentulous site following extraction. For example, the buccal-lingual/palatal dimension is changed, and the height of the buccal bone crest is decreased. Today, some people prefer a shortened post-extraction healing time or immediate implant placement following extraction. It has been suggested that the placement of an implant in the fresh extraction socket may counteract alveolar ridge contraction following tooth removal. FIGS. 10-13 illustrate such an improved procedure of tooth extraction immediately followed by a dental implantation. As shown in FIG. 10, a first reference 110a is affixed to a tooth to be extracted. Then an oral navigation base is obtained the first reference being affixed to the tooth. In the procedure, the implant is placed in the same surgical intervention as the dental extraction. As shown in FIG. 11, when the surgery starts, a second reference 110b is affixed to the jaw bone invasively, and registered to the first reference 110a or to the oral navigation base. Shortly after the tooth is extracted as shown in FIG. 12, the implantation procedure is carried out, and is guided by the oral navigation base using the second reference 110b. As shown in FIG. 13, an implant is placed in the socket of extracted tooth. The immediate implant placement exhibits some advantages including reduction of treatment time, fewer surgical interventions and a decrease in surgical trauma to the soft tissues at the implant site.

Techniques and technologies may be described herein in terms of functional and/or logical block components or modules such as various modules as shown in FIG. 1, and with reference to symbolic representations of operations, processing tasks, and functions that may be performed by various computing components or devices. Such operations, tasks, and functions are sometimes referred to as being computer-executed, computerized, processor-executed, software-implemented, or computer-implemented. It should be appreciated that the various block components shown in the figures may be realized by any number of hardware, software, and/or firmware components configured to perform the specified functions. For example, an embodiment of a system or a component may employ various integrated circuit components, e.g., memory elements, digital signal processing elements, logic elements, look-up tables, or the like, which may carry out a variety of functions under the control of one or more microprocessors or other control devices.

When implemented in software or firmware, various elements of the systems described herein are essentially the code segments or executable instructions that, when executed by one or more processor devices, cause the host computing system to perform the various tasks. In certain embodiments, the program or code segments are stored in a tangible processor-readable medium, which may include any medium that can store or transfer information. Examples of suitable forms of non-transitory and processor-readable media include an electronic circuit, a semiconductor memory device, a ROM, a flash memory, an erasable ROM (EROM), a floppy diskette, a CD-ROM, an optical disk, a hard disk, or the like.

In the foregoing specification, embodiments of the present invention have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicant to be the scope of the invention, is the literal and equivalent scope of the set of claims that issue from this application, in the specific form in which such claims issue, including any subsequent correction.

1. A stereotactic medical procedure performed on an anatomy of a patient, comprising the steps of:
(i) affixing a first reference to the anatomy;
(ii) obtaining a navigation base of the patient with the first reference being affixed to the anatomy, wherein the navigation base is image data, a coordinate system, an atlas, a morphed atlas, or any combination thereof;
(iii) affixing a second reference to the anatomy;
(iv) registering the second reference to the first reference or to the navigation base; and
(v) performing at least a part of the medical procedure guided by the navigation base using the second reference.

2. The stereotactic medical procedure according to claim 1, which is an image guided surgery, a physiological monitoring, a delivery of a medical therapy, a delivery of a medical device, or any combination thereof.

3. The stereotactic medical procedure according to claim 2, wherein the medical device is an orthopedic implant or a soft tissue implant.

4. The stereotactic medical procedure according to claim 1, wherein the first reference comprises a set of fiducial markers with a geometric shape or a material that can be recognized by a computer vision system.

5. The stereotactic medical procedure according to claim 1, wherein the first reference is affixed non-invasively to the anatomy in step (i).

6. The stereotactic medical procedure according to claim 1, wherein the navigation base is image data selected from two, three, and four dimensional images acquired from computed tomography (CT), magnetic resonance imaging (MRI), single photon emission computer tomography (SPECT), positron emission tomography (PET), ultrasound imaging (US), high frequency ultrasound (HIFU), intra-vascular ultrasound (IVUS), fluoroscopic imaging, isocentric fluoroscopy, bi-plane fluoroscopy, multi-slice computed tomography (MSCT), optical coherence tomography (OCT), or any combination thereof.

7. The stereotactic medical procedure according to claim 1, further comprising performing a portion of the medical procedure guided by the navigation base using the first reference that remains affixed, or is re-affixed, to the anatomy as it was in the step of obtaining the navigation base, before the step of affixing a second reference to the anatomy.

8. The stereotactic medical procedure according to claim 1, wherein the second reference comprises a set of fiducial markers with a geometric shape or a material that can be recognized by a computer vision system.

9. The stereotactic medical procedure according to claim 1, wherein the second reference is affixed invasively to the anatomy.

10. The stereotactic medical procedure according to claim 1, further comprising a step of removing the first reference from the anatomy after registering the second reference to the first reference or the navigation base, and before performing the medical procedure guided by the navigation base.

11. The stereotactic medical procedure according to claim 1, further comprising guiding the movement of a foreign object relative to the anatomy.

12. The stereotactic medical procedure according to claim 11, wherein the foreign object is an instrument, a tool, an implant, a medical device, a delivery system, or any combination thereof.

13. The stereotactic medical procedure according to claim 11, wherein the foreign object is a dental drill, a probe, a guide wire, an endoscope, a needle, a sensor, a stylet, a suction tube, a catheter, a balloon catheter, a lead, a stent, an insert, a capsule, a drug delivery system, a cell delivery system, a gene delivery system, an opening, an ablation tool, a biopsy window, a biopsy system, an arthroscopic system, or any combination thereof.

14. The stereotactic medical procedure according to claim 1, wherein the first reference is affixed to a site of the anatomy in step (i), and wherein the site is subject to the medical procedure performed in step (v).

15. The stereotactic medical procedure according to claim 14, wherein the site of the anatomy is a gum tissue that will be flapped for later dental implantation.

16. The stereotactic medical procedure according to claim 14, wherein the site of the anatomy is a tooth or teeth that will be extracted for later dental implantation.

17. A system used for the stereotactic medical procedure according to claim 1, comprising
two or more references for affixing to an anatomy of a patient,
a navigation base acquisition system for obtaining a navigation base of the patient with a first reference selected from the two or more references being affixed to the anatomy, wherein the navigation base is image data, a coordinate system, an atlas, a morphed atlas, or any combination thereof;
a planning module for planning the stereotactic medical procedure;
a foreign object that is foreign to, and moves relative to, the anatomy;
a tracking system for tracking both the anatomy and the foreign object;
a translation module for registering the data obtained by the tracking system in the planning module;
a superimposing module for overlaying the anatomy and the foreign object as tracked by the tracking system; and
a cascading module for shifting the reliance on one reference to the reliance on another, during the stereotactic medical procedure.

18. The system according to claim 17, wherein
the first reference is a first set of fiducial markers,
the second reference is a second set of fiducial markers,
the navigation base is a CT scan of a patient with the first set of fiducial markers,
the planning module is a dental implant treatment planning system that simulates placement of implants,
the tracking system is a camera or sensor system that can recognize and track both the first set of fiducial markers and the second set of fiducial markers,
the translation module is a registration software component that registers the fiducial markers obtained by the tracking system and their counterparts obtained in the navigation base data.
